(19) United States
(12) Patent Application Publication
(10) Pub. No.: US 2005/0239884 A1
(43) Pub. Date: Oct. 27, 2005

(54) Compositions comprising hmg-coa reductase inhibitor

(76) Inventors: Andreas Meyer, Neuenburg (DE); Jorg Ogorka, Steinen (DE); Sabine Desset-Brethes, Pratteln (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/522,421

(22) PCT Filed: Jul. 24, 2003
(86) PCT No.: PCT/EP03/08179

§ 371 Date: Mar. 25, 2005

(30) Foreign Application Priority Data
Jul. 25, 2002 (GB) 0217306.0

(51) Int. Cl. A61K 9/22; A61K 31/225

(52) U.S. Cl. 514/548; 424/468

(57) ABSTRACT

The present invention relates to pharmaceutical compositions for sustained release comprising as active ingredient an HMG-CoA reductase inhibitor or a pharmaceutically acceptable salt thereof, said composition comprising an inner phase (internal) and an outer phase (external), wherein at least the outer phase comprises at least one matrix former.

The present invention relates to pharmaceutical compositions for sustained release comprising as active ingredient an HMG-CoA reductase inhibitor or a pharmaceutically acceptable salt thereof, said composition comprising an inner phase (internal) and an outer phase (external), wherein at least the outer phase comprises at least one matrix former. When using the composition according to the present invention, unexpected advantages can be demonstrated.

The term “modified”, “extended” “sustained release” hereinbefore and hereafter shall corresponds to an active ingredient that is released from the dosage form over an extended period of time, for example greater than about four hours. Preferably, the pharmaceutical compositions release less than about 80 weight percent of the active agent in the first eight hours after ingestion of the composition, with the balance of the pharmaceutically active agent being released thereafter. In preferred compositions, less than about 15 weight percent of the pharmaceutically active agent is released in the first 0.5 hour after ingestion, from about 10 to about 50 weight percent of the pharmaceutically active agent is released within about 2 hours after ingestion, and from about 40 to about 90 preferably about 40 to about 60 weight percent of the pharmaceutically active agent is released within about 6 hours after ingestion.

HMG-CoA reductase inhibitors, also called β-hydroxy-β-methylglutaryl-co-enzyme-A reductase inhibitors (and also called statins) are understood to be those active agents which may be preferably used to lower the lipid levels including cholesterol in blood and can be used e.g. for the prevention or treatment of hyperlipidemia and artheriosclerosis.

The class of HMG-Co-A reductase inhibitors comprises compounds having differing structural features.

HMG-CoA reductase inhibitor compounds are disclosed, e.g., in the following commonly assigned patents, published patent applications and publications which are all hereby incorporated herein by reference:

Specific examples of compounds disclosed in the above publications, which are HMG-CoA reductase compounds suitable to be employed as the drug active agent in the compositions of the invention, comprise the following sodium salts, or other pharmaceutically acceptable salts:
(E)-(3R,5S)-7-[2-cyclopropyl-4-(4-fluoro-phenyl)-quinolin-3-yl]-3,5-dihydroxy-hept-6-enoic acid, calcium salt;
3R,5S-(E)-7-[4-(4-fluorophenyl)-6-(1-methylethyl)-2-dimethylaminopyrimidin-5-yl]-3,5-dihydroxy-6-heptenoic acid, sodium salt;
erythro-(±)-(E)-7-[3-(4-fluorophenyl)-spiro[cyclopentane-1,1′-1H-inden]-2′-yl]-3,5-dihydroxy-6-heptenoic acid, sodium salt;
3R,5S-(E)-7-[3-(4-fluorophenyl)-1-(1-methylethyl)-indolizin-2-yl]-3,5-dihydroxy-6-heptenoic acid, sodium salt;
3R,5S-(E)-7-[3-(4-fluorophenyl)-1-(1-methylethyl)-1H-pyrrolo[2,3-b]pyridin-2-yl]-3,5-dihydroxy-6-heptenoic acid, sodium salt;
3R,5S-(E)-7-[4-(4-fluorophenyl)-2-(1-methylethyl)-quinolin-3-yl]-3,5-dihydroxy-6-heptenoic acid, sodium salt;
3R,5S-(E)-7-[1-(4-fluorophenyl)-3-(1-methylethyl)-4-oxo-1,4-dihydroquinolin-2-yl]-3,5-dihydroxy-6-heptenoic acid, sodium salt;
3R,5S-(E)-7-[4-(4-fluorophenyl)-6-(1-methylethyl)-3-methyl-1H-pyrazolo[3,4-b]pyridin-5-yl]-3,5-dihydroxy-6-heptenoic acid, sodium salt;
3R,5S-(E)-7-[3-(1-methylethyl)-5,6-diphenyl-pyridazin-4-yl]-3,5-dihydroxy-6-heptenoic acid, sodium salt;
3R,5S-(E)-7-[4-(4-fluorophenyl)-6-(1-methylethyl)-2-phenyl-pyrimidin-5-yl]-3,5-dihydroxy-6-heptenoic acid, sodium salt;
3R,5S-(E)-7-[4-(4-fluorophenyl)-1-(1-methylethyl)-3-phenyl-2-oxo-2,3-dihydroimidazol-5-yl]-3,5-dihydroxy-6-heptenoic acid, sodium salt;
3R,5S-(E)-7-[4-(4-fluorophenyl)-2-(1-methylethyl)-1-oxo-1,2-dihydro-quinolin-3-yl]-3,5-dihydroxy-6-heptenoic acid, sodium salt;
erythro-(±)-(E)-7-[4-(4-fluorophenyl)-2-(1-methylethyl)-quinolin-3-yl]-3,5-dihydroxy-6-heptenoic acid, sodium salt;
erythro-(±)-(E)-7-[1-(4-fluorophenyl)-3-(1-methylethyl)-pyrrolo[2,1-a]isoquinolin-2-yl]-3,5-dihydroxy-6-heptenoic acid sodium salt;
erythro-(±)-(E)-7-[4-cyclopropyl-6-(4-fluorophenyl)-2-(4-methoxyphenyl)pyrimidin-5-yl]-3,5-dihydroxy-6-heptenoic acid, sodium salt;
3R,5S-(E)-7-[4-(4-fluorophenyl)-2,6-dimethylpyrimidin-5-yl]-3,5-dihydroxy-6-heptenoic acid, sodium salt;
3R,5S-(E)-7-[4-(4-fluorophenyl)-6-methyl-2-phenyl-pyrimidin-5-yl]-3,5-dihydroxy-6-heptenoic acid, sodium salt;
3R,5S-(E)-7-[(4 (3,5-dimethylphenyl)-6-methyl-2-phenyl-pyrimidin-5-yl]-3,5-dihydroxy-6-heptenoic acid, sodium salt;
erythro-(±)-(E)-7-[3,4-bis(4-fluorophenyl)-6-(1-methylethyl)-pyridazin-5-yl]-3,5-dihydroxy-6-heptenoic acid, sodium salt;
erythro-(±)-(E)-7-[1-(4-fluorophenyl)-3-(1-methylethyl)-5-phenyl-1H-pyrrol-2-yl]-3,5-dihydroxy-6-heptenoic acid, sodium salt;
erythro-(±)-(E)-9,9-bis(4-fluorophenyl)-3,5-dihydroxy-6-(1-methyl-1H-tetrazol-5-yl)-6,8-nonadienoic acid, sodium salt;
erythro-(±)-(E)-3,5-dihydroxy-9,9-diphenyl-6,8-nonadienoic acid, sodium salt;
erythro-(±)-(E)-7-[4-(4-fluorophenyl)-1,2-bis(1-methylethyl)-3-phenylpyrrol-2-yl]-3,5-dihydroxy-6-heptenoic acid, sodium salt;
3R,5S-(E)-7-[4,5-bis(4-fluorophenyl)-2-(1-methylethyl)-1H-imidazol-1-yl]-3,5-dihydroxy-6-heptenoic acid, sodium salt;
3R,5S-(E)-7-[4-(4-fluorophenyl)-2,6-bis(1-methylethyl)-5-methoxymethyl-pyridin-3-yl]-3,5-dihydroxy-6-heptenoic acid, sodium salt;
erythro-(±)-(E)-[4-(4-fluorophenyl)-2-(1-methylethyl)-6-phenyl-pyridin-3-yl]-3,5-dihydroxy-6-heptenoic acid, sodium salt;
erythro-(÷)-(E)-[2-(4-fluorophenyl)-4,4,6,6-tetramethyl-cyclohexen-1-yl]-3,5-dihydroxy-6-heptenoic acid, sodium salt;
erythro-(±)-(E)-7-[4-(4-fluorophenyl)-2-cyclopropyl-quinolin-3-yl]-3,5-dihydroxy-6-heptenoic acid, sodium salt; and
erythro-(±)-(E)-7-[4-(4-fluorophenyl)-2-(1-methylethyl)-quinolin-3-yl]-3,5-dihydroxy-6-heptenoic acid, sodium salt.

Preferred are compounds which are selected from the group consisting of atorvastatin, cerivastatin, fluvastatin, lovastatin, nisvastatin, pitavastatin (formerly itavastatin), pravastatin, rosuvastatin, and simvastatin, or, in each case, a pharmaceutically acceptable salt thereof.

Especially preferred HMG-Co-A reductase inhibitors are those agents which have been marketed. Most preferred are atorvastatin, fluvastatin, nisvastatin, pitavastatin or simvastatin or a pharmaceutically acceptable salt thereof, in the first line pitavastain or a pharmaceutically acceptable salt thereof.

Only salts that are pharmaceutically acceptable and non-toxic are used therapeutically and those salts are therefore preferred.

The corresponding active ingredient or a pharmaceutically acceptable salt thereof may also be used in form of a solvate, such as a hydrate or including other solvents, used for crystallization.

The structure of the active agents identified hereinbefore or hereinafter by generic or trade names may be taken from the actual edition of the standard compendium “The Merck Index” or from databases, e.g. Life cycle Patents International (e.g. IMS World Publications). The corresponding content thereof is hereby incorporated by reference. Any person skilled in the art is fully enabled to identify the active agent and, based on these references, likewise enabled to manufacture and test the pharmaceutical indications and properties in standard test models, both in vitro and in vivo.

In a preferred embodiment of the present invention the amount of an HMG-CoA reductase inhibitor or pharmaceutically acceptable salt thereof is about 5 to 50% by weight of the dosage unit form, preferably about 5 to 20%, most preferably about 10 to 20% of the dosage unit form, e.g. about 10%, e.g. about 11% of the dosage unit form.

In an especially preferred embodiment of the invention the amount of an HMG-CoA reductase inhibitor or pharmaceutically acceptable salt thereof is about 1-32 mg, preferably 1-16 mg per dosage unit form, especially for fluvastatin.

Hydrophilic and/or hydrophobic components can be used as matrix former.

Hydrophilic, non-ionic, slowly swelling and gel forming polymers are employed as matrix former. These polymers exhibit different swelling characteristics and therefore different viscosities in aqueous media and form upon ingestion of the solid dosage form different diffusion barriers (the matrix) releasing the drug substance by rate-controlled diffusion of the drug substance through these diffusion barriers. A substantial amount of the released active agent may be processed efficiently at the targeted active site. The non-ionic, hydrophilic polymer is present in an amount providing sufficient strength to the gel matrix to prevent its premature degradation. The gel matrix should also be formed within a time period that is effective to prevent the premature release of the active agent.

For example, the gel matrix preferably forms within about 5 minutes after ingestion of the composition to prevent a burst of active agent prior to gel formation. It has turned out that the nonionic, hydrophilic polymer operates to decrease the rate of gel formation to an acceptable level. The non-ionic, hydrophilic polymer may be present in the pharmaceutical composition in an amount ranging from about 1 to about 80 weight percent, preferably from about 1 to about 60 weight percent, more preferably from about 15 to about 50% by weight of the dosage unit form, most preferably from about 18 to about 40% by weight of the dosage unit form.

The matrix former can be selected from the group consisting of a hydroxypropyl methyl cellulose (HPMC), polyethylene glycol, polyvinylpyrrolidone, polyvinyl alcohol, and hydrophilic polymers such as hydroxypropylcellulose and hydroxymethylcellulose.

The matrix former can furthermore be selected from the group consisting of polysaccharides such as alginate, carrageenan, scleroglucan, pullulan, dextran, haluronic acid, chitin, chitosan and starch.

The matrix former can furthermore be selected from the group consisting of natural polymers such as proteins, for example, albumin or gelatine, and natural rubber.

The matrix former can furthermore be selected from the group consisting of synthetic polymers such as acrylates, for example, polymethacrylate, poly(hydroxy ethyl methacrylate), poly(methyl methacrylate), poly(hydroxy ethyl methacrylate-co-methyl metacrylate, Carbopol 934™, polamides such as polyacrylamide or poly(methylene bis acrylamide), polyanhydrides such as poly(biscarboxyphenoxy)methane, PEO-PPO block-co-polymers such as poloxamers, polyvinylchloride, polyvinyl pyrrolidone, polyvinyl alcohol, polyethylene, polyethylene glycols and co-polymers thereof, polyethylene oxides and co-polymers thereof, polypropylene and co-polymers thereof, polystyrene, polyesters such as poly(lactic acid), poly(glycolic acid), poly(caprolactone) and co-polymers thereof, poly(ortho esters and co-polymers thereof, resins such as Dowex™ or Amberlite™, polycarbonate, cellophane, silicones such as poly(dimethylsiloxane), polyurethanes, and synthetic rubbers such as styrene butadiene rubber or isopropene rubber.

The matrix former can furthermore be selected from the group consisting of shellacs, waxes such as carnauba wax, beeswax, glycowax or castor wax, nylon, stearates such as glycerol palmitostearate, glyceroyl monostearate, glyceryl tristearate or stearyl alcohol, lipids such as glycerides or phospholipids, and paraffin.

In a most preferred embodiment of the present invention an HPMC is selected as matrix former.

In a preferred embodiment of the present invention the pharmaceutical compositions comprise from about 1 to about 60% of by weight HPMC of the dosage unit form, preferably from about 15 to 50% of by weight HPMC of the dosage unit form, more preferably from about 18 to about 40% of by weight HPMC of the dosage unit form.

The HPCM components have an average molecular weight ranging from approximately 20'000 to approximately 170'000. These molecular weights might correspond to viscosities of approximately 1 to approximately 100'000 cps (viscosities values given of 2% aqueous solutions of the HPMC types.).

According to the invention, the matrix former of the internal and/or external phase may comprise one or more type(s) of matrix former(s) having different viscosities in each phase.

Preferably, the matrix former of the external phase comprises one or more type of matrix former component having different viscosities.

In a preferred embodiment of the present invention the matrix former of the inner phase has a viscosity of about 1 to about 500 cps, preferably of about 1 to about 250 cps, more preferably of about 1 to about 125 cps.

In a preferred embodiment of the present invention the matrix former of the external phase has a viscosity of about 100 to about 100000 cps, preferably of about 100 to 50000 cps, more preferably of about 100 to 25000 cps.

Furthermore the invention relates to a corresponding composition, wherein one type of the matrix former component of the external phase has a viscosity of about 80 to 150 cps and another type of matrix former component has a viscosity of about 50000 to 100000 cps.

In a preferred embodiment the invention the viscosities of the HPMC polymer(s) used as matrix former in the external phase range from approximately 100 to approximately 100'000 cps.

According to the invention, one type of the matrix former component of the external phase has a viscosity of approx. 100 cps and the other type of matrix former component has a viscosity of approx. 100'000 cps.

In a preferred embodiment, the total amount of the matrix forming HPMC component(s) present in the internal phase, having a viscosity of about 100 cps ranges from about 0-35 mg, preferably from about 10-35 mg per dosage unit form.

In a preferred embodiment, the total amount of the matrix forming HPMC component(s) present in the external phase, having a viscosity of about 100'000 cps ranges from about 10-35 mg per dosage unit form.

In another especially embodiment of the invention, the matrix forming HPMC components are selected from the group consisting of HPMC K100LVP CR 100 cps used in the internal and/or external phase (also named Methocel K100 Premium LVCR EP (100 cps) and HPMC 100T and HPMC K100LVP CR used in the external phase (also named K100M Premium CR EP (100000 cps))

The ratio between HPMC polymers contained in the “internal phase” (granulate) and the external phase, i.e., excipients admixed to the granulate after the drying/screening process is comprised between 0:100 and 100:0, preferably from about 0:50 to about 15:15, e.g 0:30; 0:15; 15:15 when comparing the amounts of the components.

The composition according to the present invention furthermore may also comprise a stabilizer, especially for protecting the drug substance adequately against pH-related destabilization.

Additionally, the heat and light sensitivity as well as hygroscopicity of an active ingredient impose particular requirements on the manufacture and storage of pharmaceutical dosage forms.

Certain HMG-CoA reductase inhibitors are extremely susceptible to degradation at pH below about 8. An example of such a compound comprises the compound having the USAN designation fluvastatin sodium (hereinafter “fluvastatin”), of the chemical designation: R*,S*-(E)-(±)-7-[3-(4-fluorophenyl)-1-(1-methyl-ethyl)-1H-indol-2-yl]-3,5-dihydroxy-6-heptenoic acid, sodium salt, [see European Patent Application EP-A-114027].

For example, the degradation kinetics of fluvastatin in aqueous solution at various pH is illustrated below:

% fluvastatin remaining at 37° C.

| pH | after 1 hour | after 24 hrs |
|---|---|---|
| 7.8 | 98.3 | 98.0 |
| 6.0 | 99.6 | 97.1 |
| 4.0 | 86.7 | 25.2 |
| 1.0 | 10.9 | 0 |

The above-indicated instability of fluvastatin and related HMG-CoA reductase compounds is believed to be due to the extreme lability of the β, δ-hydroxy groups on the heptenoic acid chain and the presence of the double bond, such that at neutral to acidic pH, the compounds readily undergo elimination or isomerization or oxidation reactions to form conjugated unsaturated aromatic compounds, as well as the threo isomer, the corresponding lactones, and other degradation products.

In order to achieve marketable dosage forms that meet the international quality criteria (e.g. for approval) comprising HMG-CoA reductase inhibitor compound, it is essential to adequately protect it against pH-related destabilization by using a stabilizer.

A preferred stabilizer to be used according to the present invention is an “alkaline medium”, said alkaline medium being capable of stabilizing the composition by imparting a pH of at least 8 to an aqueous solution or dispersion of the composition. Since the stabilizer is added in solution during the aqueous granulation process, it is in intimate contact with the active ingredient in the composition to achieve optimal stability of the medicament.

The term “alkaline medium” or “base” employed herein shall refer to one or more pharmaceutically acceptable substances capable of imparting a pH of at least 8, and preferably at least 9, and up to about pH 10, to an aqueous solution or dispersion of the composition of the invention. More particularly, the alkaline medium creates a “micro-pH” of at least 8 around the particles of the composition when water is adsorbed thereon or when water is added in small amounts to the composition. The alkaline medium should otherwise be inert to the composition compounds. The pH may be determined by taking a unit dosage of the composition containing e.g. 4 mg of pitavastatin or the equivalent amount of another compound and dispersing or dissolving the composition in 10 to 100 ml of water. The pharmaceutically acceptable alkaline substance(s) which comprise the alkaline medium may range from water-soluble to sparingly soluble to essentially water-insoluble.

In a preferred embodiment of the present invention, the stabilizer is a basic stabilizer selected from the group consisting of inorganic water-soluble or inorganic water-insoluble compound.

An inorganic water-soluble compound is a suitable carbonate salt such as sodium or potassium carbonate, sodium bicarbonate, potassium hydrogen carbonate, phosphate salts selected from, e.g., anhydrous sodium, potassium or calcium dibasic phosphate, trisodium phosphate, alkali metal hydroxides, selected from sodium, potassium, or lithium hydroxide, and mixtures thereof.

Sodium bicarbonate advantageously serves to neutralize acidic groups in the composition in the presence of moisture that may adsorb onto particles of the composition during storage.

The calcium carbonate exerts a buffering action in the stored composition, without apparent effect on drug release upon ingestion. It has further been found that the carbonate salts sufficiently stabilize the drug substance such that conventional water-based preparative techniques, e.g. trituration with water or wet granulation, can be utilized to prepare stabilized compositions of the invention.

Examples of water-insoluble compound are suitable alkaline compounds capable of imparting the requisite basicity include certain pharmaceutically acceptable inorganic compounds commonly employed in antacid compositions (e.g., magnesium oxide, hydroxide or carbonate; magnesium hydrogen carbonate; aluminum or calcium hydroxide or carbonate; composite aluminum-magnesium compounds, such as magnesium aluminum hydroxide); as well as pharmaceutically acceptable salts of phosphoric acid such as tribasic calcium phosphate; and mixtures thereof.

In a preferred embodiment of the invention, the stabilizer is an inorganic water insoluble suitable silicate compound such as magnesium aluminium silicate (neusilin). Said stabilizer can be introduced in the manufacturing process in the internal phase or in the external phase. Studies showed that neusilin has a higher stabilizing effect than some inorganic water-soluble stabilizers.

The proportion of a particularly stabilizing excipient to be employed will depend to some extent on the intended manufacturing process. In compositions to be tableted, for example, calcium carbonate should not exceed a proportion which can no longer be conveniently subjected to compression, and will generally be used in combination with a more readily compressible alkaline substance, e.g., sodium bicarbonate. On the other hand, capsule dosage forms may comprise higher levels of poorly compressible excipients, provided that the overall composition remains sufficiently free-flowing and processible.

In a preferred embodiment, the amount of the stabilizer is about 1-15 weight % of the composition.

In a preferred embodiment, the amount of stabilizer is from about 0.1-10 mg per dosage unit.

An example of a stabilized composition according to the invention may comprise: 0.1 to 60 weight % (wt. %), typically 0.5 to 40 wt. %, of the active ingredient (e.g., pitavastatin); and preferably 0.1 to 35 wt. %, more preferably 1-15 wt. % (e.g. 1 wt %, 1,25 wt %, 2 wt %, 3 wt %), of water insoluble compound such as neusilin or soluble carbonate compound, for example, selected from potassium bicarbonate, potassium carbonate and/or mixtures thereof.

It is a further advantage that the stabilized compositions of the invention can be readily prepared by aqueous or other solvent-based techniques, e.g. wet granulation.

The resulting composition has been found to provide an extended storage life of the HMG-CoA reductase inhibitor compounds, even in the presence of moisture or when such compositions additionally comprise otherwise potentially reactive excipients, such as lactose. The stability of the drug substance in compositions of the invention can be at least 95%, and is typically between 98% and 99%, after 18 months at 25° C., and for even longer periods. Compositions also having particularly attractive storage stability comprise, as an alkaline medium, both a water-soluble alkaline excipient and a water-insoluble or sparingly soluble alkaline excipient.

A solid unit dosage composition may have the ratio of water insoluble to soluble carbonate carbonate from e.g. 40:1 to 1:2.

An exemplary tablet of the invention may comprise a ratio between calcium carbonate and sodium bicarbonate of about 2:1 to 1:2 by weight. A capsule composition may comprise these excipients in a ratio of, for example, 25:1 to 35:1 by weight.

The composition according to the present invention may furthermore comprise a filler. In addition to the drug substance and alkaline medium, a filler is also generally employed in the compositions to impart processability. Suitable filler materials are well-known to the art (see, e.g., Remington's Pharmaceutical Sciences, 18th Ed. (1990), Mack Publishing Co., Easton, Pa., pp. 1635-1636), and include microcrystalline cellulose, lactose and other carbohydrates, starch, pregelatinized starch, e.g., starch 1500R (Colorcon Corp.), corn starch, dicalcium phosphate, potassium bicarbonate, sodium bicarbonate, cellulose, calcium phosphate dibasic anhydrous, sugars, sodium chloride, and mixtures thereof, of which lactose, microcrystalline cellulose, pregelatinized starch, and mixtures thereof, are preferred. Owing to its superior disintegration and compression properties, microcrystalline cellulose (Avicel PH1, Avicel R, FMC Corp.), and mixtures comprising microcrystalline cellulose and one or more additional fillers, e.g., pregelatinized starch, are particularly useful. The total filler is present in the compositions in an amount of about 1 to 65 wt. %, based on the total composition, preferably 20 to 60 wt %, more preferably 50 wt %. The invention relates to compositions wherein the total amount of the filler is from about 20-60 mg, preferably from about 2040 mg per dosage unit and preferably consists of microcrystalline cellulose.

The composition according to the present invention may furthermore comprise film coating components.

Enteric film coating components may optionally be applied to oral tablets, pellets or capsules to protect against premature degradation of the drug substance by gastric acid prior to reaching the intestinal absorption site. Examples of such materials are well-known and include hydroxypropylmethylcellulose phthalate, cellulose acetate phthalate, polyvinyl acetate phthalate, methylcellulose phthalate, copolymerized methacrylic acid/methacrylic acid methyl esters (e.g., EudragitR, Rohm Pharma). The enteric coating is preferably applied to result in about a 5 to 12, preferably 8 to 10, weight percent increase of the capsule, pellet or tablet core. Tableted compositions of the invention are desirably coated to protect against moisture and light discoloration, and to mask the bitter taste of the drug. Either the enteric coating may contain opacifiers and colorants, or a conventional opaque film coating may be applied to the tablet core, optionally after it has been coated with an enteric substance.

Examples of suitable film formers in film coating compositions to be applied to compositions of the invention comprise, e.g., polyethylene glycol, polyvinylpyrrolidone, polyvinyl alcohol, hydrophilic polymers such as hydroxypropylcellulose, hydroxymethylcellulose, and hydroxypropylmethylcellulose or the like, of which hydroxypropylmethylcellulose (e.g., Opadry YellowT, Colorcon Corp.) is preferred. Hydrophobic film-formers which may be applied using an organic solvent vehicle comprise, for example, ethyl cellulose, cellulose acetate, polyvinyl alcohol-maleic anhydride copolymers, etc.

The film coating may be generally applied to achieve a weight increase of the pellet or core or tablet of about 1 to 10 wt. %, and preferably about 2 to 6 wt. %.

Other conventional enteric or film coating composition ingredients include plasticizers, e.g., polyethylene glycol (e.g. polyethylene glycol 6000), triethylcitrate, diethyl phthalate, propylene glycol, glycerin, butyl phthalate, in conventional amounts, as well as the above-mentioned opacifiers such as titanium dioxide, and colorants, e.g. iron oxide, aluminum lakes, etc.

The enteric or film coatings can be applied by conventional techniques in a suitable coating pan or fluidized bed apparatus using water and/or conventional organic solvents (e.g., methyl alcohol, ethyl alcohol, isopropyl alcohol), ketones (acetone, ethylmethyl ketone), chlorinated hydrocarbons (methylene chloride, dichloroethane), etc.

The composition according to the present invention may furthermore comprise further components.

Further components which may be incorporated into the compositions to facilitate processing and/or provide enhanced properties of the product dosage form, are selected from the group consisting of:
a) well-known tableting binders (e.g., hydroxypropylmethylcellulose, starch, starch pregelatinized (starch 1500), gelatin, sugars, natural and synthetic gums, such as carboxymethylcellulose, methylcellulose, polyvinylpyrrolidone, low substituted hydroxypropylcellulose, ethylcellulose, polyvinylacetate, polyacrylates, gelatin, natural and synthetic gums), microcrystalline cellulose, and mixtures of the foregoing;
b) disintegrants (e.g., cross-linked carboxymethyl-cellulose, croscarmelose, crospovidone, sodium starch glycolate);
c) lubricants (e.g., magnesium stearate, stearic acid, calcium stearate, glyceryl behenate, hydrogenated vegetable oil, carnauba wax and the like);
d) flow agents (e.g., silicon dioxide, talc, polyethylene oxides);
e) anti-adherents or glidants (e.g., talc)
f) sweeteners;
g) coloring mediums (e.g., iron oxide, aluminum lakes);
h) flavoring mediums;
i) antioxidants, etc.

Selection of a particular ingredient or ingredients and the amounts used will be readily determinable by one skilled in the art by reference to standard procedures and practices for preparing tableted or encapsulated or other dosage forms. In general, an effective amount of a tableting binder will comprise about 1 to 10 wt. %, and preferably 1 to 5 wt. %; anti-adherents or glidants, about 1 to 10 wt. %; disintegrants, about 1 to 5 wt. %, and lubricants, about 0.1 to 2 wt. %, based on the total composition.

A composition according to the invention comprises (in weight percent based on the total composition):
Drug substance: approx. 5-50 wt % of the formulation; preferably 5-20 wt %, for example 10-20 wt %, e.g. about 10 wt %, e.g. about 11 wt %.
Matrix former: The amount of HPMC as matrix former is between 1 to 80 wt %, preferably between 15 and 50 wt %, more preferably 18-40 wt %
Stabilizer (alkaline medium): 1-15 wt %
Filler: About 1 to 65 wt %, preferably about 20-60 wt %, more preferably approx. 50 wt %.

The inner phase of the pharmaceutical composition according to the invention can comprise the drug substance, a filler, a binder a stabilizer, and optionally a matrix former.

The outer phase of the pharmaceutical composition according to the invention can comprise at least a matrix former agent, a flow agent, a lubricant, and optionally a filler.

In a preferred embodiment the drug substance consists in pitavastatin Ca-salt. The drug substance is used preferably at about 10%, e.g. 10.45 wt % (by weight of the dosage unit form).

In a preferred embodiment the filler consists in microcrystalline cellulose. The total amount of filler is used preferably at about 50% by weight of the dosage unit form. In a most preferred embodiment, the filler of the internal phase is used at about 20-52 wt % e.g. at about 26,05 wt %, about 39,8 wt %, about 44,8 wt %, about 46, 67 wt %, or about 51,05 wt % (by weight of the dosage unit form). In a most preferred embodiment, the filler of the external phase is used at about 15-20 wt %, e.g. 18,75 wt %.

In a preferred embodiment the binder consists in low substituted hydroxypropylcellulose HPC or hydroxypropylmethylcellulose HPMC (e.g. 3 or 6 cps). In a preferred embodiment, the binder is used at about 1-10 wt %, e.g. 10 wt %, most preferably 1-5 wt %, e.g. at about 3, 125 wt %, or 5 wt %.

In a preferred embodiment the stabilizer consists in potassium bicarbonate or magnesium aluminium metasilicate (neusilin). In a preferred embodiment the stabilizer is used at about 1-15 wt % by weight of the dosage unit form e.g. at about 1.25 wt %.

In a preferred embodiment the matrix former of the internal phase consist in HPMC having a viscosity of about 100 cps and used at about 15-40 wt %. In a most preferred embodiment the matrix former of the internal phase is used at about 18,75 wt %, about 31,25 wt %, or about 37,5 wt %, by weight of the dosage unit form.

In a preferred embodiment the matrix former of the external phase consist in HPMC. According to the invention, one type of the matrix former component of the external phase has a viscosity of approx. 100 cps and the other type of matrix former component has a viscosity of approx. 100'000 cps.

In a most preferred embodiment the matrix former of the external phase having a viscosity of approx 100 cps is used at about 15-40 wt %, e.g. at about 18.75 wt % or about 37,5 wt % by weight of the dosage unit form.

In a most preferred embodiment the matrix former of the external phase having a viscosity of approx. 100'000 cps is used at about 15-40 wt %, e.g. at about 18.75 wt % or about 37,5 wt % by weight of the dosage unit form.

In a preferred embodiment the flow agent consists in silicon dioxide colloidal (e.g. Aerosil). In a preferred embodiment the flow agent is used at about 0.1-2 wt %, e.g. 0.5 wt %.

In a preferred embodiment the lubricant consists in magnesium stearate. In a preferred embodiment the lubricant is used at about 0.1-2 wt %, e.g. 0.5 wt %.

According to the invention, there is provided a composition, wherein the ratio between the matrix former in the internal and external phase is:
a) from about 0:30 to about 15:15, e.g. 0:30; 0:15; 15:15, when comparing the amount of the components (in mg).
b) from about 0:38 to about 18,75:18,75, e.g 0:37,5; 0:18,75; 18,75:18,75, when comparing the weight percent of the components.

The present invention relates to compositions wherein the ratio between the total HPMC 100 cps and HPMC 100'000 cps of the outer phase is
a) from about 0:30 to about 30:0, e.g. 0:30; 0:15; 15:15; 15:0; 25:0; 30:0, when comparing the amount of the components (in mg).
b) from about 0:38 to about 38:0, e.g. 0:37,5; 0:18,75; 0:18,75; 18,75:18,75: 18,75:0; 37,5:0, when comparing the weight percent of the components.

Furthermore the invention relates to a composition wherein the ratio between HPMC 100 cps of the internal phase and HPMC 100'000 cps of the external phase is:
a) from about 0:30 to about 30:0, e.g 0:30; 0:15; 15:15; 15:0; 25:0; 30:0 when comparing the amount of the components (in mg).
b) from about 0:38 to about 38:0, e.g. 0:37,5; 18,75:18,75; 18,75:0; 31,25:0; 37,5:0 when comparing the weight percent of the components.

The invention particularly relates to compositions wherein the ratio between the matrix forming HPMC in the internal phase and the total weight, is:
a) from about 0:80 to about 30:80, e.g. 0:80; 15:80; 25:80; 30:80 when comparing the amount of the components (in mg).
b) from about 0:100 to about 38:100, e.g. 0:100; 18,75:100; 31,25:100; 37,5:100 when comparing the weight percent of the components.

The invention particularly relates to compositions wherein the ratio between the matrix forming HPMC in the external phase and the total weight is:
a) from about 0 to about 30:80, preferably from about 15:80 to about 30:80, e.g 15:80; 30:80 when comparing the amount of the components (in mg).
b) from about 18:100 to a bout 38:100, e.g 18,75:100; 37,5:100 when comparing the weight percent of the components.

The invention particularly relates to compositons wherein the ratio between the total amount of matrix forming HPMC and the total weight, is:
a) from about 15:80, to about 30:80 e.g 15:80; 25:80; 30:80; when comparing the amount of the components (in mg).
b) from about 18:100 to a bout 38:100, e.g 18,75:100; 37,5:100 when comparing the weight percent of the components.

The present invention is concerned with compositions wherein the ratio between the matrix forming HPMC total and the HMG-CoA reductase inhibitor is
a) from about 15:8,36 to about 30:8,36, e.g 15:8,36; 30:8,36 when comparing the amount of the components (in mg).
b) from about 18, 75:10,45 to about 37,5:10,45, e.g 18, 75:10,45; 37,5:10,45 when comparing the weight percent of the components.

According to the invention, there are provided compositions wherein the ratio between the matrix forming HPMC in the internal phase and the HMG-CoA reductase inhibitor is
a) from about 0 to approx. 6/1, preferably from about 0 to about 30:8, e.g 15:8,36; 25:8,36; 30:8,36 when comparing the amount of the components (in mg).
b) from about 0:10,45 to about 38:10.45, e.g. 0:10,45; 18,75:10,45; 31,25:10,45; 37,5:10,45 when comparing the weight percent of the components.

According to the invention, there are provided compositions wherein the ratio between the matrix forming HPMC in the external phase and the HMG-CoA reductase inhibitor is
a) from about 0 to about 30:8, e.g 15:8,36; 30:8,36 when comparing the amount of the components (in mg).
b) From about 0 to about 37,5:10,45 preferably 18,75:10,45 to about 37,5:10,45, e.g 18,75:10,45; 37,5:10,45 when comparing the weight percent of the components.

According to the invention, there is provided a composition, wherein the ratio between the matrix former in the internal and external phase is
a) from about 0:30 to about 30:0, e.g 0:30; 0:15; 15:15; 25:0; 30:0 when comparing the amount of the components (in mg).
b) from about 0:38 to about 16:16, e.g 0:37,5; 0:18,75; 18,75:18,75; 31,25:0; 37,5:0 when comparing the weight percent of the components.

Furthermore the invention relates to a composition wherein the ratio between the filler in the internal phase and the matrix former HPMC comprised in the internal phase is
a) from about 35:0 to about 40:30, e.g. 37,34:0; 35,84:15; 35,80:30; 20,84:15;31,84:15; 33,64:15; 40,84:25 when comparing the amount of the components (in mg).
b) from about 47:0 to about 40:19, e.g. 46,67:0; 44,8:18,75; 44,8:37,5; 42,05:18,75; 51,05:31.25; 26,05:18,75; 39,8:18,75 when comparing the weight percent of the components.

The invention particularly relates to a composition wherein the ratio between the total matrix former HPMC in the internal phase and the total weight, is
a) from about 0:80 to about 30:80, e.g 0:80; 15:80; 25:80; 30:80 when comparing the amount of the components (in mg).
b) from about 0:100 to about 37,5:100, e.g. 0:100; 18,75:100; 31,25:100; 37,5:100 when comparing the weight percent of the components.

The invention particularly relates to a composition wherein the ratio between the total matrix former HPMC in the external phase and the total weight is
a) from about 15:80 to about 30:80, e.g, 15:80; 30:80 when comparing the amount of the components (in mg).
b) From about 18,75:100 to about 37,5:100, e.g 0:100; 18,75:100; 37,5:100 when comparing the weight percent of the components.

The invention particularly relates to a composition wherein the ratio between the total matrix former HPMC and the total weight is
a) from about 15:80 to about 30:80, e.g. 15:80; 25:80; 30:80 when comparing the amount of the components (in mg)
b) from about 18,75:100 to about 37,5:100, e.g 18,75:100; 31,25:100; 37,5:100 when comparing the weight percent of the components.

According to the invention, there is provided a composition wherein the ratio between the HPMC3 cps intern and the HMG-CoA reductase inhibitor is.
a) from about 2/9 to about 4/8, e.g 2,5:8,36; 4:8,36 when comparing the amount of the components (in mg)
b) From about 3:10 to about 6:10, e.g 3,125:10,45; 5:10,45 when comparing the weight percent of the components.

The present invention is concerned with a composition wherein the ratio between the HPMC100 cps intern and the HMG-CoA reductase inhibitor is
a) from about 0:9 to about 30:9, e.g 0:8,36; 15:8,36; 25:8.36; 30:8.36 when comparing the amount of the components (in mg).
b) from about 0:10,45 to about 37,5:10.45, e.g 0:10,45; 18,75:10,45; 31,25:10,45; 37,5:10,45 when comparing the weight percent of the components.

The present invention is concerned with a composition wherein the ratio between the matrix former HPMC total and the HMG-CoA reductase inhibitor is:
a) from about 15:9 to about 30:9, e.g 15:8,36; 25:8,36; 30:8,36 when comparing the amount of the components (in mg).
b) from about 18,75:10,45 to about 37,5:10,45, e.g 18,75:10,45; 31,25:10,45; 37,5:10,45 when comparing the weight percent of the components.

The present invention is concerned with a composition wherein the ratio between the filler in the internal phase and the matrix former HPMC comprised in the internal phase is
a) from about 37:0 to about 41:25, e.g 37,34:0; 35,84:15; 33,64:15; 20,84:15; 31,84:15; 40,84:25; 35,84:30 when comparing the amount of the components (in mg).
b) from about 46:0 to about 42:19, e.g 46,67:0; 44,8:18,75; 44,8:37,5; 51,05:31,25; 26,05:18,75; 39,8:18,75; 42,05:18,75 when comparing the weight percent of the components.

The invention relates to a composition wherein the ratio between the filler in the external phase and the matrix former HPMC comprised in the external phase is
a) from about 0:30 to about 15:15, e.g 0:30; 0:15; 15:15 when comparing the amount of the components (in mg).
b) from about 0:37,5 to about 18,75:18,75, e.g 0:37,5; 0:18,75; 18,75:18,75 when comparing the weight percent of the components.

The invention relates to a composition wherein the ratio between the filler in the internal phase and the matrix former HPMC total is
a) from about 20:30 to about 38:15, e.g 20,84:30; 31,84:30; 33,64:30; 35,84:30; 37,34:30; 40,84:25; 37,34:15 when comparing the amount of the components (in mg).
b) from about 26:37 to about 47:19, eg. 26,05:37,5; 39,8:37,7; 42,05:37,5; 46,67:37,5; 44,8:37,5; 51,05:31,25; 46,67:18,75 when comparing the weight percent of the components.

The invention also relates to a composition wherein the ratio between the filler in the external phase and the matrix former HPMC total is
a) from about 0:30 to about 15:15, e.g 0:30; 0:25; 15:30; 15:15 when comparing the amount of the components (in mg).
b) from about 0:37,5 to about 18,75:18,75, e.g 0:37,5; 0:31,25; 18,75:37,5; 18,75:18,75 when comparing the weight percent of the components.

It has been surprisingly found that the composition according to the invention more advantageously increases the distribution of the HMG-CoA reductase inhibitor to the liver due to the slow drug release and decreases the drug plasma levels and consequently the distribution to the muscle tissue. The consequence is a better tolerability as compared to the tolerability of the same dose of an immediate release composition of the HMG-CoA reductase inhibitor. Because of the improved tolerability of the extended release composition higher doses can be administered leading to higher efficacy of the drug. The improved tolerability of the pharmaceutical composition and consequently higher efficacy, due to the possibility to administer higher doses, according to the invention is based on a well adaptated extended release profile. An improved adapted extended release profile is due notably to the presence of the matrix former of different viscosities in both the inner and external phase of the composition according to the present invention and is also due to the adequate distribution between the inner and/or outer (external) phase, creating an advantageous diffusion barrier by hydrogel formation of the matrix in aqueous media. Furthermore, a small size of the pharmaceutical dosage form and, in parallel, the possibility to apply a low dose formulation of active ingredient induce a better tolerability of the active ingredient.

To obtain very stable compositions, an aqueous or other solvent-based preparative process is preferably utilized, whereby the drug substance and alkaline medium are blended together in the presence of minor amounts of, e.g., water, to provide particles containing the drug and alkaline substance in intimate admixture. The solvent or liquid dispersion medium can be for example, water, ethanol, a polyol (for example, glycerol, propylene glycol, liquid polyethylene glycols, and the like), vegetable oils, nontoxic glyceryl esters, and suitable mixtures thereof. Given the hygroscopicity and moisture sensitivity of HMG-CoA reductase inhibitor compounds such as fluvastatin, it is unexpected that the drug substance is sufficiently stabilized by the alkaline medium to resist degradation by a such techniques.

In another embodiment of a solvent-based process which can assist subsequent drying in a fluidized bed, the drug substance and alkaline medium are wet granulated by known techniques, i.e. blended in the moistened state, together with an amount of the filler material. The thus-formed granules, after drying, are then combined with any remaining filler and other set-asides, e.g., binder, lubricant, and can therefore be tableted, encapsulated, or otherwise shaped into a dosage form. Drying is conventionally performed by tray drying or in a fluidized bed, preferably the latter.

It has been found that a water-soluble stabilizing alkaline substance such as sodium carbonate or bicarbonate or other alkaline medium, can be added insitu to the above-described aqueous phase comprising the fluvastatin or other HMG-CoA reductase inhibitor compound, and upon subjecting this aqueous phase to a freeze-drying procedure, there can be obtained particles comprising the drug compound co-lyophilized with the added alkaline substance. Very good contacting of the drug and stabilizer can thereby be achieved, to the extent that stable compositions of the invention may be prepared, for example, from the drug and sodium carbonate in a weight ratio of about 10/1 to 100/1. For example, a co-lyophilized composition of the invention comprising as low as 0.1% by weight sodium carbonate has been found effective to provide a highly stabilized drug composition.

As previously indicated, an enteric and/or film coating composition can be applied to the dosage form for its particular benefits. Enteric or film coating of a microcrystalline cellulose-based tablet with a water-based film coating composition is desirably carried out at a bed temperature of 30-50° C., an inlet temperature of 50-80° C. and a relative humidity (RH) of less than 50%.

The resulting tableted or capsule dosage forms should be protected during storage against thermal or light induced oxidation as well as moisture contamination.

Pharmaceutical compositions, e.g. oral dosage forms, according to the invention may be formulated in any conventional form, e.g. powders, granules/granulates, capsules or tablets. Preferred pharmaceutical compositions may be in the form of tablets.

The pharmaceutical composition according to the invention may have a dosage weight from about 5 to about 300 mg, preferably about 100 mg, more preferably about 80 mg.

Such compositions may be formulated by known means to provide standard unitary oral dosages of compound, e.g., 4 mg, 8 mg, 12 mg, 16 mg, etc., as e.g., powders, granulates, capsules, pellets or tablets.

A special embodiment of the invention relates to tablet having a diameter from 4 to 8 mm, for example from 6 to 8 mm having a weight between 70 to 180 mg wherein the active ingredient has a weight between 4 and 40 mg per dosage unit form.

Pharmaceutical compositions, e.g. oral dosage forms, hereinabove described may be formed of a granulated mass comprising fluvastatin, HPMC and optionally other exciplents commonly used in pharmaceutical composition, e.g. oral dosage forms, e.g. tablets.

Various dissolution profiles of different strengths can therefore be obtained either by compressing the same tabletting mixture to tablets of dose proportional weights or by maintaining the same tablet size/weight over all dosage strengths (weight compensation by the excipient used as filler).

The pharmaceutical compositions according to the invention can be prepared by use of well known pharmaceutical processing techniques such as blending, granulation, milling, spray drying, compaction, or coating.
A generic manufacturing procedure of the pharmaceutical composition, e.g. oral dosage forms can be described in the following steps:
   Step 1: Place the drug substance, the matrix former(s) (or combinations of them), the disintegrant(s) (if requested) and the filler(s) (if requested, also further components as listed on page 13) into the bowl of the high shear mixer (remark: the matrix former may be omitted, according the the actual composition).
   Step 2: Mix (e.g., 5 minutes)
   Step 3: Dissolve the stabilizer in purified water
   Step 4: Add the solution to the mixture of step (2)
   Step 5: Rinse the container of step (3) with purified water and add the rinsing liquid to the mixture of step (4)
   Step 6: Mix/knead/granulate the compounds.
   Step 7: Screen the wet granulate (e.g., a sieve of 2 mm mesh size).
   Step 8: Dry the granulate on trays or in a fluid bed dryer (preferred).
   Step 9: Screen the dried granulate into the container of a free fall mixer (e.g., a sieve of 1 mm mesh size).
   Step 10: Mix the matrix former(s), filler(s), disintegrant(s), glidant(s)/flow agent(s) (if requested, also further components as listed on page 13) in the free fall mixer.
   Step 11: Screen the lubricant(s) to the mixture of step (10) or prepare a premix of the lubricant(s) with a small part of the mixture (10) and screen this lubricant(s) premix to the remaining part of mixture (10).
   Step 12: Mix the components of step (11).
   Step 13: Compress the tabletting mixture of step (12) on a force feeding (rotary) tabletting machine to tablets (or tablet cores, if film coating is necessary) of the required weight and dimensions.
   Step 14: (optional) Add the film formers to the required liquid (solvents mixtures) or purified water) and dissolve the film former. Add plasticizer(s), if required.
   Step 15: (optional) Prepare a suspension of the coloring agent(s) and titanium dioxide (white pigment) in the required liquid.
   Step 16: (optional) Add the suspension of step (15) to the solution of step (14).
   Step 17: (optional) Stir the mixture until a homogeneous dispersion/solution is obtained
   Step 18: (optional) Spray the suspension of step (17) on the cores of step (13) until the required weight of the film coat is achieved.

Said process can be generalized as follow:
mixture of components comprising the drug substance and the matrix former
addition of a stabilizer
formation of a granulate
compression of the granulate to form a tablet or a tablet core
optional: addition of a film coating comprising a film former
   In a preferred embodiment, the manufacturing procedure of the pharmaceutical composition, e.g. oral dosage forms, using Potassium bicarbonate as stabilizer and HPMC as matrix former can, for example, be described in the following steps:
      Step 1: Place the drug substance, HPMC (binder, low viscosity), HPMC or different HPMC qualities (matrix former, high viscosity) and microcrystalline cellulose (powder) into the bowl of the high shear mixer (remark: the matrix former may be omitted, according the the actual composition).
      Step 2: Mix (e.g., 5 minutes)
      Step 3: Dissolve Potassium bicarbonate in purified water
      Step 4: Add the solution to the mixture (2)
      Step 5: Rinse the container of step (3) with purified water and add the rinsing liquid to the mixture of step (4)
      Step 6: Mix/knead/granulate the compounds.
      Step 7: Screen the wet granulate (e.g., a sieve of 2 mm mesh size).
      Step 8: Dry the granulate on trays or in a fluid bed dryer (preferred).
      Step 9: Screen the dried granulate into the container of a free fall mixer (e.g., a sieve of 1 mm mesh size).
      Step 10: Mix HPMC or different HPMC qualities (matrix former, high viscosity), microcrystalline cellulose (granular) and colloidal silicon dioxide in the free fall mixer (remark: the microcrystalline cellulose (granular) may be omitted according to the actual composition).
      Step 11: Screen magnesium stearate to the mixture of step (10).
      Step 12: Mix the components of step (11).
      Step 13: Compress the tabletting mixture of step (12) on a force feeding (rotary) tabletting machine to tablets (or tablet cores, if film coating is necessary) of the required weight and dimensions.
      Step 14: (optional) Add the prepared dry powder blend for the film coat preparation (e.g., Opadry) to purified water
      Step 15: (optional) Stir the mixture until a homogeneous dispersion/solution is obtained
      Step 16: (optional) Spray the suspension of step (15) on the cores of step (13) until the required weight of the film coat is achieved.
   An alternative (generic) manufacturing procedure of the pharmaceutical composition, e.g. oral dosage forms using neusilin as stabilizer can be described in the following steps:
      Step 1: Place the drug substance, the matrix former(s) (or combinations of them), the disintegrant(s) (if requested), the Neusilin and the filler(s) (if requested, also further components as listed on page 13) into the bowl of the high shear mixer (remark: the matrix former may be omitted, according the the actual composition).
      Step 2: Mix (e.g., 5 minutes)
      Step 3: Add the solution to the mixture of step (2)
      Step 4: Mix/knead/granulate the compounds.
      Step 5: Screen the wet granulate (e.g., a sieve of 2 mm mesh size).
      Step 6: Dry the granulate on trays or in a fluid bed dryer (preferred).
      Step 7: Screen the dried granulate Into the container of a free fall mixer (e.g., a sieve of 1 mm mesh size).
      Step 8: Mix the matrix former(s), filler(s), disintegrant(s), glidant(s)/flow agent(s) (if requested, also further components as listed on page 13) in the free fall mixer.
      Step 9: Screen the lubricant(s) to the mixture of step (8) or prepare a premix of the lubricant(s) with a small part of the mixture (9) and screen this lubricant(s) premix to the remaining part of mixture (8).
      Step 10: Mix the components of step (9).
      Step 11: Compress the tabletting mixture of step (10) on a force feeding (rotary) tabletting machine to tablets (or tablet cores, if film coating is necessary) of the required weight and dimensions.
      Step 12: (optional) Add the film formers to the required liquid (solvent(s mixtures) or purified water) and dissolve the film former. Add plasticizer(s), if required.
      Step 13: (optional) Prepare a suspension of the coloring agent(s) and titanium dioxide (white pigment) in the required liquid.
      Step 14: (optional) Add the suspension of step (13) to the solution of step (12).
      Step 15: (optional) Stir the mixture until a homogeneous dispersion/solution is obtained
      Step 16: (optional) Spray the suspension of step (15) on the cores of step (11) until the required weight of the film coat is achieved.

Said process can be generalized as follow:
mixture of components comprising the drug substance the matrix former and the stabilizer
formation of a granulate
compression of the granulate to form a tablet or a tablet core
optional: addition of a film coating comprising a film former
   In an other preferred embodiment, the pharmaceutical compositions according to the invention can be prepared by use of well known pharmaceutical processing techniques such as blending, granulation, milling, spray drying, compaction, or coating, e.g. the manufacturing procedure of the pharmaceutical composition, e.g. oral dosage forms, using HPMC as matrix former and neusilin as stabilizer can, for example, be described in the following steps:
      Step 1: Place the drug substance, HPMC (binder, low viscosity), HPMC or different HPMC qualities (matrix former, high viscosity), microcrystalline cellulose (powder) and Neusilin into the bowl of the high shear mixer (remark: the matrix former may be omitted, according to the actual composition).
      Step 2: Mix (e.g., 5 minutes)
      Step 3: Add the granulating liquid to the mixture of step (2)
      Step 4: Mix/knead/granulate the compounds.
      Step 5: Screen the wet granulate (e.g., a sieve of 2 mm mesh size).
      Step 6: Dry the granulate on trays or in a fluid bed dryer (preferred).
      Step 7: Screen the dried granulate into the container of a free fall mixer (e.g., a sieve of 1 mm mesh size).
      Step 8: Mix HPMC or different HPMC qualifies (matrix former, high viscosity), microcrystalline cellulose (granular) and colloidal silicon dioxide in the free fall mixer (remark: the microcrystalline cellulose (granular) may be omitted according to the actual composition).
      Step 9: Screen magnesium stearate to the mixture of step (8).
      Step 10: Mix the components of step (9).
      Step 11: Compress the tabletting mixture of step (10) on a force feeding (rotary) tabletting machine to tablets (or tablet cores, if film coating is necessary) of the required weight and dimensions.
      Step 12: (optional) Add the prepared dry powder blend for the film coat preparation (e.g., Opadry) to purified water.
      Step 13: (optional) Stir the mixture until a homogeneous dispersion/solution is obtained
      Step 14: (optional) Spray the suspension of step (13) on the cores of step (11) until the required weight of the film coat is achieved.

The following examples are intended to illustrate the invention in various of its embodiments without being limitative in anyway thereof.

### EXAMPLE 1

Inner phase: 10.45 wt % of drug substance, for example pitavastatin Ca-salt, 46.67 wt % of microcrystalline cellulose, 3.13 wt % of HPMC (3 cps), 1.25 wt % of inorganic water-soluble compound (such as potassium bicarbonate) or water insoluble compound (such as neusilin), the external phase comprising 37.5 wt % HPMC (100'000 cps), 0.5 wt % of silicium dioxide colloidal and 0.5 wt % of magnesium stearate.

### EXAMPLE 1

### BIS

Inner phase: 10.45 wt % of drug substance, for example pitavastatin Ca-salt, 46.675 wt % of microcrystalline cellulose, 3.125 wt % of HPMC (3 cps), 1.25 wt % of inorganic water-soluble compound (such as potassium bicarbonate) or water insoluble compound (such as neusilin), the external phase comprising 37.5 wt % HPMC (100'000 cps), 0.5 wt % of silicium dioxide colloidal and 0.5 wt % of magnesium stearate.

### EXAMPLE 1

### TER

Inner phase: 8.36 mg of drug substance, for example pitavastatin Ca-salt, 37.34 mg of microcrystalline cellulose, 2.5 mg of HPMC (3 cps), 1 mg of inorganic water-soluble compound (such as potassium bicarbonate) or water insoluble compound (such as neusilin), the external phase comprising 30 mg of HPMC (100'000 cps), 0.4 mg of silicium dioxide colloidal and 0.4 mg of magnesium stearate.

### EXAMPLE 2

Inner phase: 10.45 wt % of drug substance-alkaline medium, for example pitavastatin Ca-salt, 46.67 wt % of microcrystalline cellulose, 3.13 wt % of HPMC (3 cps), 1,25 wt % of inorganic water-soluble compound (such as potassium bicarbonate) or water insoluble compound (such as neusilin), the external phase comprising 18.75 wt % HPMC (100'000 cps), 18.75 wt % HPMC (100 cps), 0.5 wt % of silicium dioxide colloidal and 0.5 wt % of magnesium stearate.

### EXAMPLE 2

### BIS

Inner phase: 10.45 wt % of drug substance, for example pitavastatin Ca-salt, 46.675 wt % of microcrystalline cellulose, 3.125 wt % of HPMC (3 cps), 1,25 wt % of inorganic water-soluble compound (such as potassium bicarbonate) or water insoluble compound (such as neusilin), the external phase comprising 18.75 wt % HPMC (100'000 cps), 18.75 wt % HPMC (100 cps), 0.5 wt % of silicium dioxide colloidal and 0.5 wt % of magnesium stearate.

### EXAMPLE 2

### TER

Inner phase: 8.36 mg of drug substance, for example pitavastatin Ca-salt, 37.34 mg of microcrystalline cellulose, 2.5 mg of HPMC (3 cps), 1 mg of inorganic water-soluble compound (such as potassium bicarbonate) or water insoluble compound (such as neusilin), the external phase comprising 15 mg of HPMC (100'000 cps), 15 mg of HPMC (100 cps), 0.4 mg of silicium dioxide colloidal and 0.4 mg of magnesium stearate.

### EXAMPLE 3

Inner phase: 10.45 wt % of drug substance-alkaline medium, for example pitavastatin Ca-salt, 46.67 wt % of microcrystalline cellulose, 3.13 wt % of HPMC (3 cps), 1,25 wt % of inorganic water-soluble compound (such as potassium bicarbonate) or water insoluble compound (such as neusilin), the external phase comprising 37.5% HPMC (100 cps), 0.5 wt % of silicium dioxide colloidal and 0.5 wt % of magnesium stearate.

### EXAMPLE 3

### BIS

Inner phase: 10.45 wt % of drug substance, for example pitavastatin Ca-salt, 46.675 wt % of microcrystalline cellulose, 3.125 wt % of HPMC (3 cps), 1,25 wt % of inorganic water-soluble compound (such as potassium bicarbonate) or water insoluble compound (such as neusilin), the external phase comprising 37.5% HPMC (100 cps), 0.5 wt % of silicium dioxide colloidal and 0.5 wt % of magnesium stearate.

### EXAMPLE 3

### TER

Inner phase: 8.36 mg of drug substance, for example pitavastatin Ca-salt, 37.34 mg of microcrystalline cellulose, 2.5 mg of HPMC (3 cps), 1 mg of inorganic water-soluble compound (such as potassium bicarbonate) or water insoluble compound (such as neusilin), the external phase comprising 30 mg HPMC (100 cps), 0.4 mg of silicium dioxide colloidal and 0.4 mg of magnesium stearate.

### EXAMPLE 4

Inner phase: 10.45 wt % of drug substance, for example pitavastatin Ca-salt, 44.8 wt % of microcrystalline cellulose, 5 wt % of HPMC (3 cps), 18.75 wt % HPMC (100 cps), 1,25 wt % of inorganic water-soluble compound (such as potassium bicarbonate) or water insoluble compound (such as neusilin), the external phase comprising 18.75 wt % HPMC (100'000 cps), 0.5 wt % of silicium dioxide colloidal and 0.5 wt % of magnesium stearate.

### EXAMPLE 4

### BIS

Inner phase: 8.36 mg of drug substance, for example pitavastatin Ca-salt, 35.84 mg of microcrystalline cellulose, 4 mg of HPMC (3 cps), 15 mg of HPMC (100 cps), 1 mg of inorganic water-soluble compound (such as potassium bicarbonate) or water insoluble compound (such as neusilin), the external phase comprising 15 mg of HPMC (100'000 cps), 0.4 mg of silicium dioxide colloidal and 0.4 mg of magnesium stearate.

### EXAMPLE 5

Inner phase: 10.45 wt % of drug substance, for example pitavastatin Ca-salt, 44.8 wt % of microcrystalline cellulose, 5 wt % of HPMC (3 cps), 37.5 wt % % HPMC (100 cps) 1,25 wt % of inorganic water-soluble compound (such as potassium bicarbonate) or water insoluble compound (such as neusilin), the external phase comprising 0.5 wt % of silicium dioxide colloidal and 0.5 wt % of magnesium stearate.

### EXAMPLE 5

### BIS

Inner phase: 8.36 mg of drug substance, for example pitavastatin Ca-salt, 35.84 mg of microcrystalline cellulose, 4 mg of HPMC (3 cps), 30 mg of HPMC (100 cps), 1 mg of inorganic water-soluble compound (such as potassium bicarbonate) or water insoluble compound (such as neusilin), the external phase comprising 0.4 mg of silicium dioxide colloidal and 0.4 mg of magnesium stearate.

### EXAMPLE 6

Inner phase: 10.45 wt % of drug substance, for example pitavastatin Ca-salt, 51.05 wt % of microcrystalline cellulose, 5 wt % of HPMC (3 cps), 31.25 wt % HPMC (100 cps) 1,25 wt % of inorganic water-soluble compound (such as potassium bicarbonate) or water insoluble compound (such as neusilin), the external phase comprising 0.5 wt % of silicium dioxide colloidal and 0.5 wt % of magnesium stearate.

### EXAMPLE 6

### BIS

Inner phase: 8.36 mg of drug substance, for example pitavastatin Ca-salt, 40.84 mg of microcrystalline cellulose, 4 mg of HPMC (3 cps), 25 mg of HPMC (100 cps), 1 mg of inorganic water-soluble compound (such as potassium bicarbonate) or water insoluble compound (such as neusilin), the external phase comprising 0.4 mg of silicium dioxide colloidal and 0.4 mg of magnesium stearate.

### EXAMPLE 7

Inner phase: 10.45 wt % of drug substance, for example pitavastatin Ca-salt, 26.05 wt % of microcrystalline cellulose, 5 wt % of HPMC (3 cps), 18.75 wt % HPMC (100 cps) 1,25 wt of inorganic water-soluble compound (such as potassium bicarbonate) or water insoluble compound (such as neusilin), the external phase comprising 18.75 wt % HPMC (100'000 cps), 18.75 wt % of microcrystalline cellulose, 0.5 wt % of silicium dioxide colloidal and 0.5 wt % of magnesium stearate.

### EXAMPLE 7

### BIS

Inner phase: 8.36 mg of drug substance, for example pitavastatin Ca-salt, 20.84 mg of microcrystalline cellulose, 4 mg of HPMC (3 cps), 15 mg of HPMC (100 cps), 1 of inorganic water-soluble compound (such as potassium bicarbonate) or water insoluble compound (such as neusilin), the external phase comprising 15 mg of HPMC (100'000 cps), 15 mg of microcrystalline cellulose, 0.4 mg of silicium dioxide colloidal and 0.4 mg of magnesium stearate.

### EXAMPLE 8

Inner phase: 10.45 wt % of drug substance, for example pitavastatin Ca-salt, 26.05 wt % of microcrystalline cellulose, 5 wt % of HPMC (3 cps), 18.75 wt % HPMC (100 cps) 1,25 wt % of inorganic water-soluble compound (such as potassium bicarbonate) or water insoluble compound (such as neusilin), the external phase comprising 18.75 wt % HPMC (100 cps), 18.75 wt % of microcrystalline cellulose, 0.5 wt % of silicium dioxide colloidal and 0.5 wt % of magnesium stearate.

### EXAMPLE 8

### BIS

Inner phase: 8.36 mg of drug substance, for example pitavastatin Ca-salt, 20.84 mg of microcrystalline cellulose, 4 mg of HPMC (3 cps), 15 mg HPMC (100 cps) 1 mg of inorganic water-soluble compound (such as potassium bicarbonate) or water insoluble compound (such as neusilin), the external phase comprising 15 mg HPMC (100 cps), 15 mg of microcrystalline cellulose, 0.4 mg of silicium dioxide colloidal and 0.4 mg of magnesium stearate.

### EXAMPLE 9

Inner phase: 10.45 wt % of drug substance, for example pitavastatin Ca-salt, 39.8 wt % of microcrystalline cellulose, 5 wt % of HPMC (3 cps), 5 wt % of HPC, 18.75 wt % HPMC (100 cps) 1,25 wt % of inorganic water-soluble compound (such as potassium bicarbonate) or water insoluble compound (such as neusilin), the external phase comprising 18.75 wt % HPMC (100'000 cps), 0.5 wt % of silicium dioxide colloidal and 0.5 wt % of magnesium stearate.

### EXAMPLE 9

### BIS

Inner phase: 8.36 mg of drug substance, for example pitavastatin Ca-salt, 31.84 mg of microcrystalline cellulose, 4 mg of HPMC (3 cps), 4 mg of HPC, 15 mg of HPMC (100 cps) 1 mg of inorganic water-soluble compound (such as potassium bicarbonate) or water insoluble compound (such as neusilin), the external phase comprising 15 mg HPMC (100'000 cps), 0.4 mg of silicium dioxide colloidal and 0.4 mg of magnesium stearate.

### EXAMPLE 10

Inner phase: 10.45 wt % of drug substance, for example pitavastatin Ca-salt, 39.8 wt % of microcrystalline cellulose, 5 wt % of HPMC (3 cps), 5 wt % of HPC, 18.75 wt % HPMC (100 cps), 1,25 wt % of inorganic water-soluble compound (such as potassium bicarbonate) or water insoluble compound (such as neusilin), the external phase comprising 18.75 wt % HPMC (100 cps), 0.5 wt % of silicium dioxide colloidal and 0.5 wt % of magnesium stearate.

### EXAMPLE 10

### BIS

Inner phase: 8.36 mg of drug substance, for example pitavastatin Ca-salt, 31.84 mg of microcrystalline cellulose, 4 mg of HPMC (3 cps), 4 mg of HPC, 15 mg HPMC (100 cps), 1 mg of inorganic water-soluble compound (such as potassium bicarbonate) or water insoluble compound (such as neusilin), the external phase comprising 15 mg HPMC (100 cps), 0.4 mg of silicium dioxide colloidal and 0.4 mg of magnesium stearate.

### EXAMPLE 11

Inner phase: 10.45 wt % of drug substance-alkaline medium, for example pitavastatin Ca-salt, 46.67 wt % of microcrystalline cellulose, 3.13 wt % of HPMC (3 cps), 1,25 wt % of inorganic water-soluble compound (such as potassium bicarbonate) or water insoluble compound (such as neusilin), the external phase comprising 18.75 wt % HPMC (100'000 cps), 18.75 wt % microcrystalline cellulose, 0.5 wt % of silicium dioxide colloidal and 0.5 wt % of magnesium stearate.

### EXAMPLE 11

### BIS

Inner phase: 10.45 wt % of drug substance, for example pitavastatin Ca-salt, 46.675 wt % of microcrystalline cellulose, 3.125 wt % of HPMC (3 cps), 1,25 of inorganic water-soluble compound (such as potassium bicarbonate) or water insoluble compound (such as neusilin) wt %, the external phase comprising 18.75 wt % HPMC (100'000 cps), 18.75 wt % microcrystalline cellulose, 0.5 wt % of silicium dioxide colloidal and 0.5 wt % of magnesium stearate.

### EXAMPLE 11

### TER

Inner phase: 8.36 mg of drug substance, for example pitavastatin Ca-salt, 37.34 mg of microcrystalline cellulose, 2.5 mg of HPMC (3 cps), 1 mg of inorganic water-soluble compound (such as potassium bicarbonate) or water insoluble compound (such as neusilin), the external phase comprising 15 mg of HPMC (100'000 cps), 15 mg of microcrystalline cellulose, 0.4 mg of silicium dioxide colloidal and 0.4 mg of magnesium stearate.

### EXAMPLE 12

Inner phase: 10.45 wt % of drug substance-alkaline medium, for example pitavastatin Ca-salt, 46.67 wt % of microcrystalline cellulose, 3.13 wt % of HPMC (3 cps), 1,25 wt % of inorganic water-soluble compound (such as potassium bicarbonate) or water insoluble compound (such as neusilin), the external phase comprising 18.75 wt % HPMC (100 cps), 18.75 wt % microcrystalline cellulose, 0.5 wt % of silicium dioxide colloidal and 0.5 wt % of magnesium stearate.

### EXAMPLE 12

### BIS

Inner phase: 10.45 wt % of drug substance, for example pitavastatin Ca-salt, 46.675 wt % of microcrystalline cellulose, 3.125 wt % of HPMC (3 cps), 1,25 wt % of inorganic water-soluble compound (such as potassium bicarbonate) or water insoluble compound (such as neusilin), the external phase comprising 18.75 wt % HPMC (100 cps), 18.75 wt % microcrystalline cellulose, 0.5 wt % of silicium dioxide colloidal and 0.5 wt % of magnesium stearate.

### EXAMPLE 12

### TER

Inner phase: 8.36 mg of drug substance, for example pitavastatin Ca-salt, 37.37 mg of microcrystalline cellulose, 2.5 mg of HPMC (3 cps), 1 mg of inorganic water-soluble compound (such as potassium bicarbonate) or water insoluble compound (such as neusilin), the external phase comprising 15 mg of HPMC (100 cps), 15 mg of microcrystalline cellulose, 0.4 mg of silicium dioxide colloidal and 0.4 mg of magnesium stearate.

### EXAMPLE 13

Inner phase: 10.45 wt % of drug substance, for example pitavastatin Ca-salt, 42.05 wt % of microcrystalline cellulose, 5 wt % of HPMC (3 cps), 18.75 wt % of HPMC (100 cps), 4 wt % of Neusilin, the external phase comprising 18.75 wt % HPMC (100 000 cps), 0.5 wt % of silicium dioxide colloidal and 0.5 wt % of magnesium stearate.

### EXAMPLE 13

### BIS

Inner phase: 8.36 mg of drug substance, for example pitavastatin Ca-salt, 33.640 mg of microcrystalline cellulose, 4 mg of HPMC (3 cps), 15 mg of HPMC (100 cps), 3.2 mg of Neusilin, the external phase comprising 15 mg of HPMC (100 000 cps), 0.4 mg of silicium dioxide colloidal and 0.4 mg of magnesium stearate.

The examples which showed a preferred release profile are examples 1 Bis, 2 Bis, 4,7,8,9,11 Bis and 13. Most preferred examples are example 4 and example 13.

The present invention also relates to a pharmaceutical composition for the treatment of hyperlipidemia, hypercholesterolemia and atherosclerosis, as well as other diseases or conditions in which HMG-CoA reductase is implicated comprising an HMG-CoA reductase inhibitor or a pharmaceutically acceptable salt thereof and a matrix former, wherein said composition comprises an internal and an external phase wherein at least the outer phase comprises a matrix former.

The present invention also relates to a method of treatment of hyperlipidemia, hypercholesterolemia and atherosclerosis, as well as other diseases or conditions in which HMG-CoA reductase is implicated comprising administering to a patient in need thereof a therapeutically effective amount of a composition according to the invention.

The present invention also concerns a method of releasing a pharmaceutically active agent in a mammal, wherein the method includes orally administering the pharmaceutically active agent to the mammal as part of a composition according to the invention.

The present invention also concerns a pharmaceutical composition for the treatment of hyperlipidemia, hypercholesterolemia and atherosclerosis, as well as other diseases or conditions in which HMG-CoA reductase is implicated comprising an HMG-CoA reductase inhibitor or a pharmaceutically acceptable salt thereof and a matrix former, wherein said composition comprises an internal and an external phase wherein at least the outer phase comprises a matrix former.

The present invention also concerns the use of the composition according to the invention in the manufacture of a medicament for use in the treatment or prevention of a cardiovascular disease, e.g., hypercholesterolemia, hyperproteinemia and/or atherosclerosis.

In a preferred embodiment the invention relates to the use of the composition according to the invention in the manufacture of a medicament wherein said medicament is a hypercholesteremic, hyperlipoproteinemic or anti-atherosclerotic agent.

1. A pharmaceutical composition for sustained release comprising as active ingredient an HMG-CoA reductase inhibitor or a pharmaceutically acceptable salt thereof, said composition comprising an inner phase (internal) and an outer phase (external), wherein at least the outer phase comprises at least one matrix former.

2. A composition according to claim 1, wherein the HMG-CoA reductase inhibitor is selected from the group consisting of atorvastatin, cerivastatin, fluvastatin, lovastatin, pitavastatin, pravastatin, rosuvastatin, and simvastatin, or, in each case, a pharmaceutically acceptable salt thereof.

3. A composition according to claim 2, wherein the HMG-CoA reductase inhibitor is pitavastatin or a pharmaceutically acceptable salt thereof.

4. A composition according claim 1, wherein the amount of HMG-CoA reductase inhibitor or pharmaceutically acceptable salt thereof is about 5-50 weight % of the composition.

5. A composition according to claim 1, wherein the amount of HMG-CoA reductase inhibitor or pharmaceutically acceptable salt thereof is about 1-32 mg.

6. A composition according to claim 1, wherein the inner phase comprises a matrix former.

7. A composition according to claim 6, wherein the matrix former of the inner phase comprises one or more types of matrix former components having different viscosities.

8. A composition according to claim 7, wherein the matrix former of the inner phase has a viscosity of about 1 to about 500 cps.

9. A composition according to claim 1, wherein the matrix former of the external phase comprises one or more type of matrix former component having different viscosities.

10. A composition according to claim 9, wherein the matrix former of the external phase has a viscosity of about 100 to about 100000 cps.

11. A composition according to claim 1, wherein the matrix former is selected from the group consisting of polyethylene glycol, polyvinylpyrrolidone, polyvinyl alcohol, hydrophilic polymers such as hydroxypropylcellulose, hydroxymethylcellulose, and hydroxypropylmethylcellulose or the like.

12. A composition according to claim 11, wherein the matrix former is hydroxypropylmethylcellulose (HPMC).

13. A composition according to claim 12 wherein the amount of HPMC as a matrix former is about 1-60 weight % of the composition.

14. A composition according to claim 1, wherein said composition further comprises a stabilizer.

15. A composition according to claim 14, wherein the stabilizer is magnesium aluminium metasilicate (neusilin).

16. A composition according to claim 14, wherein the amount of the stabilizer is about 1-15 weight % of the composition.

17. A method of treatment of hyperlipidemia, hypercholesterolemia and atherosclerosis, as well as other diseases or conditions in which HMG-CoA reductase is implicated comprising administering to a patient in need thereof a therapeutically effective amount of a composition according to claim 1.

18. Use of the composition according to claim 1 in the manufacture of a medicament for use in the treatment or prevention of a cardiovascular disease, e.g., hypercholesterolemia, hyperproteinemia and/or atherosclerosis.

19. A composition according claim 3, wherein the amount of HMG-CoA reductase inhibitor or pharmaceutically acceptable salt thereof is about 5-50 weight % of the composition

20. A composition according to claim 3, wherein the amount of HMG-CoA reductase inhibitor or pharmaceutically acceptable salt thereof is about 5-50 weight % of the composition

21. A composition according to claim 3, wherein the amount of HMG-CoA reductase inhibitor or pharmaceutically acceptable salt thereof is about 1-32 mg.

22. A composition according to claim 3, wherein the inner phase comprises a matrix former

23. A composition according to claim 3, wherein the matrix former of the external phase comprises one or more type of matrix former component having different viscosities.

24. A composition according to claim 3, wherein the matrix former is selected from the group consisting of polyethylene glycol, polyvinylpyrrolidone, polyvinyl alcohol, hydrophilic polymers such as hydroxypropylcellulose, hydroxymethylcellulose, and hydroxypropylmethylcellulose or the like.
